(19) United States
(12) Patent Application Publication
(10) Pub. No.: US 2023/0301510 A1
(43) Pub. Date: Sep. 28, 2023

(54) ROBOTIC-ASSISTED OPTICAL COHERENCE TOMOGRAPHY (OCT)

(71) Applicant: Worcester Polytechnic Institute, Worcester, MA (US)

(72) Inventors: Haichong Zhang, Shrewsbury, MA (US); Yu Chen, Worcester, MA (US); Xihan Ma, Worcester, MA (US)

(21) Appl. No.: 18/125,823

(22) Filed: Mar. 24, 2023

Related U.S. Application Data
(60) Provisional application No. 63/323,181, filed on Mar. 24, 2022.

(51) Int. Cl. A61B 3/10; A61B 34/30; G06T 17/00

(52) U.S. Cl. CPC A61B 3/102; A61B 34/30; G06T 17/00

(57) ABSTRACT

A robotically driven OCT scanning and imaging system provides a 3-dimensional (3D) image rendering based on a 2-dimensional (2D) surface traversal of a Region of Interest (ROI) for immediate depiction of tissue health with an accuracy and portability not available with conventional imaging approaches. A comprehensive scan over the surface of the ROI ensures complete coverage, and a signal indicative of attenuation of the optical scan indicates penetration of the OCT stimulus. The received signal for each location is aggregated, or “stitched” together with the signal received from adjacent locations to provide a full mapping of the scanned region, and rendered as a color or shading map for showing anomalies or sudden variances in tissue health.

### RELATED APPLICATIONS

This patent application claims the benefit under 35 U.S.C. § 119(e) of U.S. Provisional Patent App. No. 63/323,181, filed Mar. 24, 2022, entitled “ROBOTIC-ASSISTED OPTICAL COHERENCE TOMOGRAPHY (OCT),” both incorporated herein by reference in entirety.

### STATEMENT OF FEDERALLY SPONSORED RESEARCH AND DEVELOPMENT

This invention was made with government support under grant R01 DK133717, awarded by the National Institute for Health (NIH). The government has certain rights in the invention

### BACKGROUND

Most medical imaging provides diagnostic views of anatomical structures in a noninvasive manner. Anatomical structures may be identified and assessed prior to any kind of invasive procedure. Such technologies generally involve a propagated wave medium that reflects or refracts off anatomical features for qualitative assessment thereof. Typical imaging technologies include Ultrasound (US), CT (Computed Tomography), MRI (Magnetic Resonance Imaging) and X-Ray, each having various features and costs. More recently, Optical Coherence Tomography (OCT) is emerging for beneficial properties of portability and high resolution.

### SUMMARY

A robotically driven OCT scanning and imaging system provides a 3-dimensional (3D) image rendering based on a 2-dimensional (2D) surface traversal of a Region of Interest (ROI) for immediate depiction of tissue health with an accuracy and portability not available with conventional imaging approaches. A comprehensive scan over the surface of the ROI ensures complete coverage, and a signal indicative of attenuation of the optical scan indicates penetration of the OCT stimulus. The received signal for each location is aggregated, or “stitched” together with the signal received from adjacent locations to provide a full mapping of the scanned region, and rendered as a color or shading map for showing anomalies or sudden variances in tissue health.

Configurations herein are based, in part, on the observation that medical imaging provides a valuable diagnostic tool for non-invasive visualization of tissue health and for locating unhealthy regions prior to more invasive procedures. Unfortunately, conventional approaches to medical scanning suffer from the shortcoming that the expense and size of certain scanning mediums limit their effectiveness in time-sensitive contexts. Accordingly, configurations herein substantially overcome the shortcomings of conventional approaches by providing an OCT scanning system for precise, portable imaging deployable in an operating room or similar specialized environment for fast, high resolution images of the ROI. A wide-field OCT system provides for biological subject inspection which enables 1) automatic wide-field OCT scan and 2) 3D visualization of the scanned area.

Optical Coherence Tomography (OCT) is a high-resolution, real-time, non-invasive medical imaging modality. OCT has been frequently adopted in skin and eye disease diagnosis. Many other emerging clinical applications include real-time OCT guidance/monitoring for endovascular procedures such as percutaneous coronary intervention (PCI) and atrial fibrillation ablation, etc. Moreover, researchers show that OCT can offer histopathological information of organs (kidney, liver, lung, etc.) that is impossible to obtain using conventional procedures.

In a particular example, organ transplant procedures are time sensitive due to the health and viability of a transplanted organ. Confirmation of organ health ensures a successful procedure, however 3D images via MRI incur a substantial time delay, and are too large to deploy in an operating room. Ultrasound may be performed on-site, but may lack the resolution needed to properly ascertain organ health. In a kidney transplant, for example, an OCT scan as disclosed herein is achievable in a timely manner, using co-located equipment with an accuracy sufficient for assessing kidney health by rendering 3D structures in the kidney indicative of the health (or absence thereof).

OCT scanning is viable for desktop usage with a limited, sub-centimeter level field of view (FOV) in lateral and elevational directions. For better clinical flexibility, portable OCT systems have emerged for hand-held manual guidance of the OCT probe. While a greater area of biological tissue can be covered using the hand-held probe, the collected OCT images still lack localization information (where the images are captured on the organ/tissue), hence it is impossible for global visualization and quantification of the interested features in locally acquired images. It would be beneficial to provide an OCT scan guided in a comprehensive manner to ensure complete assessment of the ROI.

Configurations discussed below provide a method for scanning tissue, which provides for traversing an OCT probe according to a scan path over a tissue specimen including a region of interest (ROI), the scan path including a plurality of parallel segments, and receiving a signal indicative of a tissue property of the tissue specimen along the scan path, such that the signal is based on an attenuation at a tissue location where the signal was received. An image processor coalesces a plurality of the received signals over the ROI for generating a 2-dimensional (2D) rendering of 3-dimensional (3D) structures in the tissue specimen indicative of a health of the tissue specimen.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a system context diagram of a medical diagnosis environment suitable for use with configurations herein;

FIG. 2 shows a scan path for a region of interest on a pre-transplant kidney;

FIG. 3 shows a depiction of the scanned region of the kidney of FIG. 2 based on the scan path;

FIGS. 4A and 4B show perspective and side views of the scan signal received from the scan path of FIG. 2;

FIG. 5 shows a block diagram of data flow in the system of FIG. 1 for gathering the signals of FIGS. 4A and 4B;

FIG. 6 is a flowchart of system operation for gathering the OCT signals as in FIGS. 1-5 to generate a map of the ROI (Region of Interest);

FIG. 7A shows the signal received from a scan of a vertical section (“slice”) of FIGS. 3, 4A and 4B;

FIGS. 7B and 7C show an axial attenuation mapping and a depth encoded mapping, respectively, from the scanning of FIGS. 1-7A; and

FIG. 8 shows a further example of an axial attenuation mapping covering an entire human kidney.

### DETAILED DESCRIPTION

The description below presents an example of OCT pre-transplant kidney monitoring is invoked as an example to illustrate the clinical needs for wide-field and spatially localized OCT imaging. Other clinical procedures using OCT can also benefit from this technology. With respect to pre-transplant kidney assessment application, the predominantly used clinical trial is anatomical feature-based pathological scoring obtained from a conventional biopsy. However, the biopsy only evaluates a few spots on the kidney, leading to potentially biased results. In contrast, the disclosed approach can function as a type of “optical biopsy”, providing OCT cross-sectional images of tissue morphology in situ and in real-time. In kidney transplantation, the kidney pathology can be obtained with OCT and used for pre-transplant prediction of delayed graft function (DGF), which is detrimental to the patient's survival. Studies further reveal that an increased tubular lumen diameter suggests a higher possibility of DGF. To fully capture the kidney microstructures (tubular lumens) for better prediction of kidney viability, a system with spatially resolved imaging capability is preferable.

In conventional approaches, OCT systems have traditionally been used for desktop usage with a limited, sub-centimeter level FOV in lateral and elevational directions. For better clinical flexibility, portable OCT systems are emerging, allowing physicians to hold the probe and perform the scan. While a greater area of biological tissue can be covered using the hand-held probe, the collected OCT images still lack localization information (where the images are captured on the organ/tissue), hence it is impossible for global visualization and quantification of the interested features in locally acquired images.

It is extremely challenging for existing OCT systems to scan the entire kidney. To achieve comprehensive kidney evaluation, it would be beneficial to develop a platform with trackable OCT probe pose in 3-dimension so that each OCT image can be registered to the common global coordinate frame.

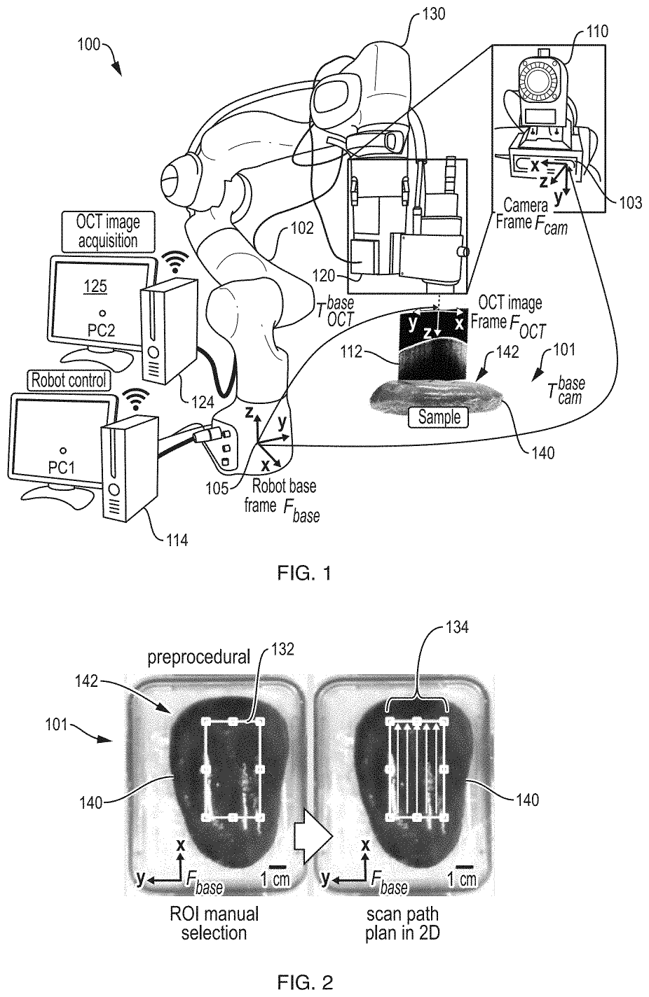

FIG. 1 is a system context diagram of a medical diagnosis environment suitable for use with configurations herein. Referring to FIG. 1, the proposed robotic-OCT system 100 captures the tissue sample placed in the designated area 101 using an RGB-depth camera 110 mounted to a 7 degree-of-freedom (DoF) robotic actuator 130 along with an OCT probe 120. Robotic control is provided by a robotic processor 114, and an OCT scanning and image processor 124 provides coalescing, or “stitching” of a signal from the OCT probe 120. A user-specified region of interest (ROI) in the camera view 112 is taken to plan the scan path that covers the ROI. A robot 102 then moves the OCT probe 120 to follow the pre-planned scan path. During the scan, the 2D OCT images and the corresponding OCT image frame 103 poses (pose stamps) relative to a robot base frame 105 are continuously recorded. Further, the height of the OCT probe 120 is automatically compensated in real-time so that the OCT image frame consistently maintains an optimal distance from the tissue surface 142 of a tissue sample 140 for imaging and diagnosis/assessment. After the scan, 3D OCT image stitching is performed using the 2D OCT images with pose stamps. 3D stitching results are visualized through depth encoding and axial attenuation mapping to demonstrate the OCT probe tracking accuracy.

FIG. 2 shows a scan path 134 for a region of interest on a pre-transplant kidney. Referring to FIGS. 1 and 2, the ROI 132 designates a portion of the surface 142 of the tissue sample 140 for analysis. While not necessarily addressing the entire surface, the ROI 132 encompasses a majority or at least enough of the tissue area to provide a health assessment. The scan path 134 defines actuated movement of the OCT probe 120 over the ROI, typically in a series of parallel runs collectively covering a rectangular region.

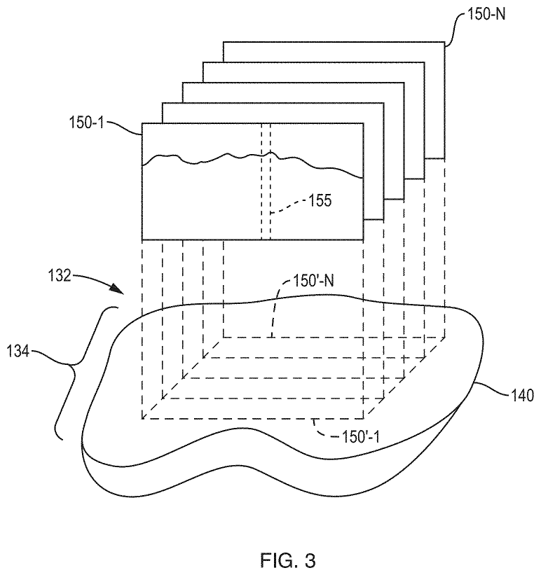

FIG. 3 shows a depiction of the scanned region of the kidney of FIG. 2 based on the scan path 134. Referring to FIGS. 1-3, the robot 102 drives the actuator 130 according to the path 134 while gathering a signal along each parallel run. The gathered signal is represented as a vertical section 150-1 . . . 150-N, or “slice” of the tissue sample 140 indicating the OCT detected properties of the tissue in the “slice.” The base frame 105 defines a Cartesian volume of the ROI based on the region of interest 132 and an orientation of the image frame 103 position of the OCT probe 120 driven by the actuator 130 directing the traversal of the OCT probe. The OCT probe 120 occupies a relative position within the Cartesian volume to allow reconciliation of the signal with a vertical section 150 of the tissue 140. From the gathered OCT signals, the OCT processor 124 forms a tissue point cloud based on the signal received at each location and the position of the OCT probe, a distance of the OCT probe 120 above a surface of the tissue specimen 140, and a position along the scan path 134. The received OCT signals may be continuous as the probe 120 moves, or may be gathered in a series of distinct points along the path 134. A rendering apparatus 125 such as a video screen then renders a map indicative of the ROI 132 based on each pixel 155 defined by a cartesian position of the ROI within the cartesian volume.

Continuing to refer to FIG. 1, FIG. 1 shows the coordinate frame convention of the robotic-OCT system. F_{base} is the robot base frame; F_{OCT} is the OCT image frame, defined at the top center of the OCT image with a constant offset (23 mm) away from the tip of the OCT probe; F_{cam} is the camera 120 color and depth image aligned frame (referred to as the camera frame). Note the x-y plane of F_{cam} and the x-y plane of F_{OCT} are parallelly assembled. T^{base} OCT is the homogeneous transformation from F_{base} to F_{OCT}; T^{base}_{cam} is the homogeneous transformation from F_{base} to F_{cam}. In terms of the hardware architecture, the example arrangement features a customized end-effector designed to mount the OCT probe and the RGB-depth camera to the robot's 7^{th} joint. The homogeneous transformation matrices from the robot base frame 105 to the OCT image frame 103 and to the camera frame are derived from the end-effector's CAD model. Two workstations are used to control the robot motion (PC1) and collect real-time 2D OCT images (PC2), respectively.

In a pre-scan phase, the tissue sample 140 is placed in the designated area 101 in the RGB-depth camera's FOV when the robot 102 is at home configuration. As kidney transplant surgery is a time-sensitive task, the boundary area of the kidney, which is hard to determine and may not contain as much information as the central area in the OCT image, can be ignored from the scan to realize higher time efficiency. Therefore, the square or rectangular shape ROI 132 with height h_{ROI} and width w_{ROI} in millimeters is drawn through a graphic user interface (GUI) to exclude the outskirt of the sample in the camera FOV.

A scan path plan is needed to ensure that the ROI will be scanned comprehensively. Orienting the y, z-axis of the OCT image frame to the opposite direction of the y, z-axis of the robot base frame at the home configuration, the scan path 134 consists of multiple scan lines, each corresponding to a pixel 155, in parallel with the x-axis of the robot base frame. This scan path 134 is formally defined as n number of lengths, or segments 150′-1 . . . 150′-N (150′ generally) and starting coordinates for the scan lines [l_{n×1}, s_{n×1}], each corresponding to a respective vertical section 150, where n is calculated based on the overlap between consecutive scan lines wₒₗ in millimeter, the lateral FOV of the OCT image w_{oct} in millimeter, and w_{ROI} as:

$$n = ⌊ \frac{{w}_{ROI}}{( {w}_{oct} - {w}_{ol} )} ⌋ , s . t . {w}_{oct} > {w}_{ol} > 0$$

All scan lines are set to be the equal length of h_{ROI}. Each starting coordinate sᵢ∈R³ is first obtained as the pixel positions in R² by distributing the scan lines along the lateral direction of the camera FOV with the left-most scan line being

$$\frac{( {w}_{OCT} {w}_{ol} )}{2}$$

away from the ROI left boundary, then projected to 3D positions relative to the camera frame utilizing the depth information and the camera intrinsic.

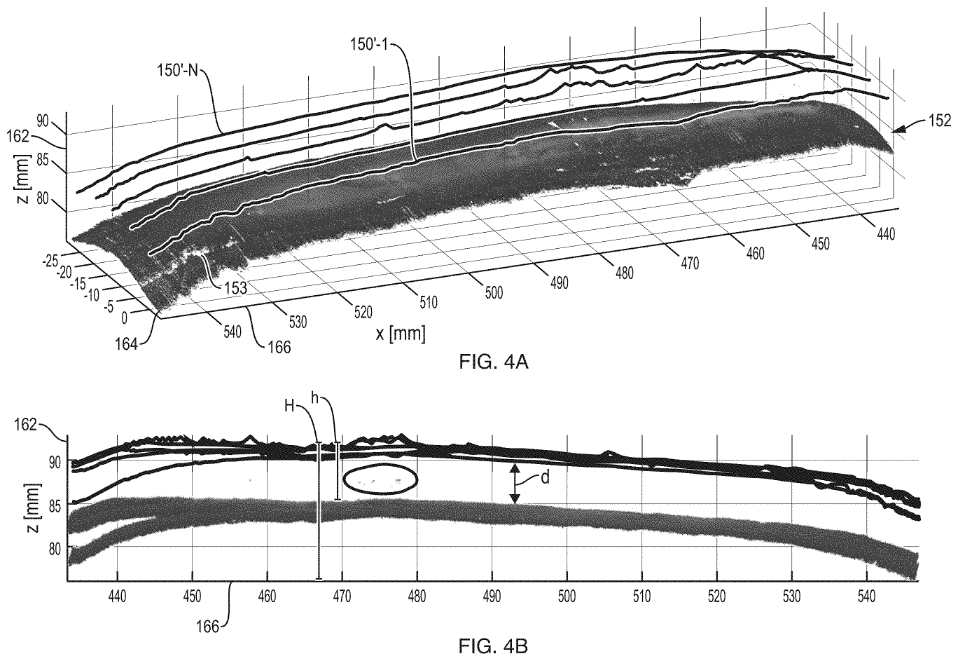

FIGS. 4A and 4B show perspective and side views of the scan signal received from the scan path of FIG. 2. Referring to FIG. 4A, a path 150′-1 . . . 150′-N is shown for each respective vertical section 150 as the robotic scan trajectory (i.e., the OCT 120) is superimposed on the raw point cloud 152, where scan line bar indicates the vertical section (e.g. 150-N) of OCT images being collected and the position of the OCT probe 120 above the tissue surface 142. The vertical axis Z 162 shows the height above the tissue specimen 140, and the lateral Y axis 164 separates the scan lines on the path 134. The OCT scanning direction given by the path 134 runs along the X axis 166. Pixel intensity in the OCT image is represented as the point cloud 152 intensity. The white region 153 suggests the pixel intensity in the correspondent OCT image is too weak to be extracted. FIG. 4B shows the side view of FIG. 4A, where d refers to the distance between the OCT image frame and the tissue surface, denoted as h above, and the total elevation above the robot 102 base as H. The OCT probe 120 traverses the tissue specimen 140 by, for each segment on the scan path, commencing a traversal of the segment by actuating the OCT probe 120 for approaching a surface of the tissue specimen on each with a landing movement that disposes the OCT probe downward to a height h based on a proportion of a distance above the tissue surface and a height H above the bottom tissue surface. Therefore the scanning motion of each segment, or vertical section 150, includes a “landing” as the OCT probe 120 descends towards the tissue surface 142 to maintain height h over the variance of the tissue surface for optimal OCT signal propagation.

Accurate execution of the path plan is beneficial for acquiring OCT images from the desired ROI. In the intra-scan procedure, the robot 102 needs to move the OCT probe 120 to each starting coordinate of the scan line and perform the scanning motion. Since the starting coordinates are initially with respect to the camera frame, the following transformation is applied to bring them to the robot base frame 105:

$$[ \begin{matrix}{s}_{1 \times n}^{base} \\ {1}_{1 \times n}\end{matrix} ] = {T}_{camera}^{base} \cdot [ \begin{matrix}{s}_{1 \times n} \\ {1}_{1 \times n}\end{matrix} ]$$

Without changing the orientation of the end-effector at the robot's home configuration, we implemented a Proportional-Derivative (PD) controller to control the Cartesian space velocity of the OCT image frame. The controller first locates the OCT probe 120 to an entry pose which is above the starting coordinate with a safety distance in the vertical direction. This step is to factor out inaccurate depth sensing of the camera, which may cause the probe to collide with the tissue if directly proceeding the robot to the starting coordinates. Next, a landing motion is designed to drop the OCT probe gradually towards the tissue at a constant linear velocity in z-axis with respect to the robot base frame. When the probe is close enough to the tissue, the tissue surface will appear in the OCT image, which can be easily captured using pixel intensity thresholding. A tissue height measurement μ is formulated to track the tissue surface in the OCT image as:

$$μ = 1 - \frac{h}{H} , s . t . H > h > 0$$

where H and h are depicted in FIG. 4B. The landing process stops when μ is greater than 65%, followed by the scan motion with a constant linear velocity (0.6 mm/s) in the scan line direction (same as the positive direction of the robot base frame's x-axis). During the scanning motion, the OCT images are sampled at 20 Hz and paired with corresponding T^{base}_{OCT} which is obtained from the robot's internal state sensing. The OCT images with pose stamps are saved from memory when the probe reaches the end of each scan line before the robot moves the probe to the subsequent entry pose and repeats the above process.

To avoid the tissue being lost in the OCT image view or making contact with the OCT probe throughout the scanning motion, a fixed distance between the OCT image frame and the tissue surface is desired, depicted by h. Hence, an automatic probe height compensation controller maintains the tissue height h at 65% by controlling the z-axis linear velocity relative to the robot base. The current velocity v_{z} at time step t is computed as:

v_{z}[t]=f·p·tan h(0.65−μ)+(1−f)·v_{z}[t−1]

where f is a constant parameter ranging from 0 to 1 such that a low pass filter is embedded to avoid velocity jittering, p is the empirically assigned proportional control gain. The tan h(*) is the hyperbolic tangent function used to guarantee the smoothness of the velocity profile.

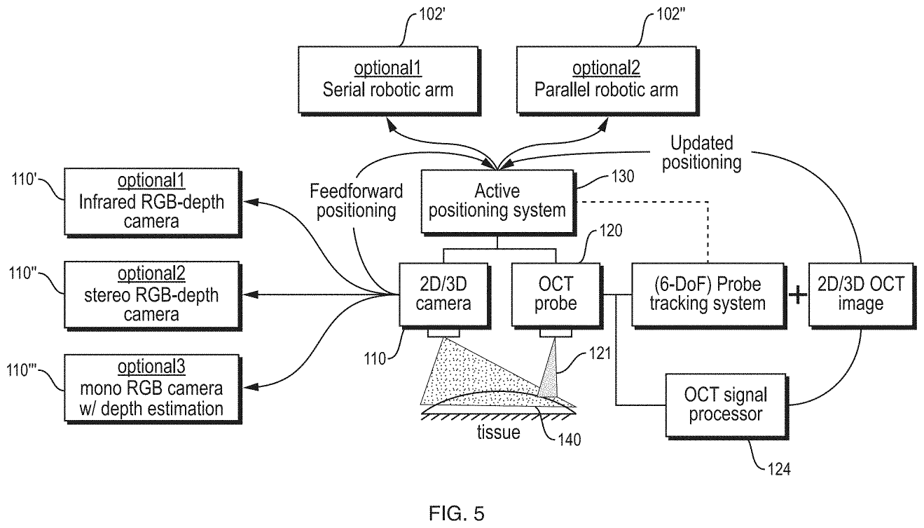

FIG. 5 shows a block diagram of data flow in the system of FIG. 1 for gathering the signals of FIGS. 4A and 4B. To realize the autonomous OCT scanning and 3D OCT imaging components, there are several implementation options. There are at least six combinations to realize the collective autonomous scanning component. The camera 110 used for scan path generation should provide depth sensing for the estimation of the relative pose between the scan subject (e.g. specimen 140) and itself, adjacent the OCT emitted optical signal 121. The range of possible options expands from an infrared/stereo RGB-depth camera 110′, a stereo RGB depth camera 110″ to mono RGB camera with algorithm-enabled depth estimation 110′″. An actuated (active) positioning mechanism is also necessary to move the OCT probe 120 for emitting an optical signal 121 along the scan path 134, which can be achieved using a serial 102′ or parallel robotic arm 102″. Parallel robots usually have higher positioning accuracy but suffer from less workspace than serial robots, hence the selection between the type of robotic arm depends on the application needs.

The serial robotic arm (active positioning) is employed as a representative design choice to demonstrate the proposed autonomous wide-field OCT imaging functionality. Furthermore, pre-transplant kidney monitoring is used as an example clinical application.

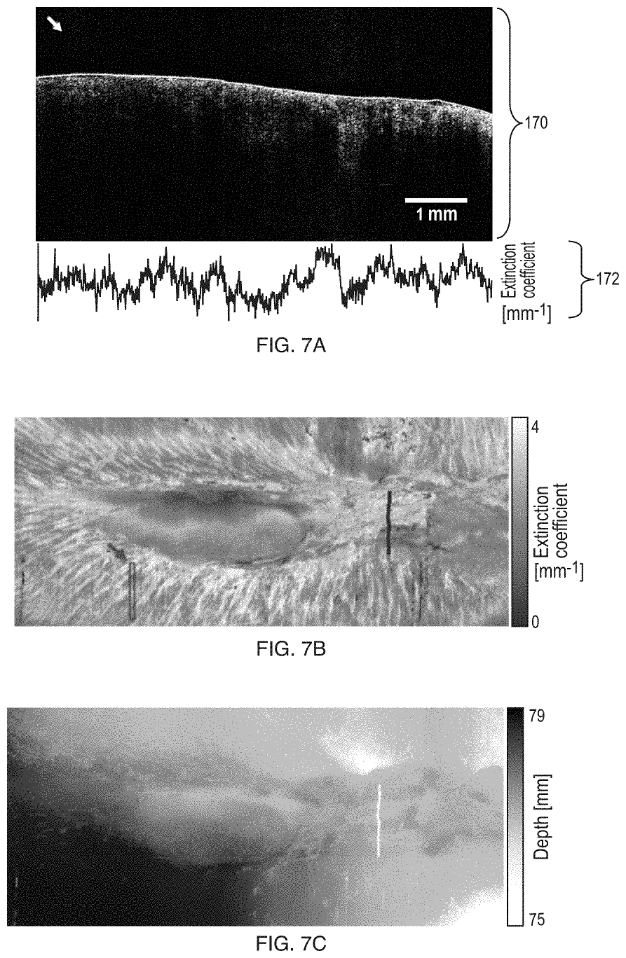

In the configurations herein, the robot 102 and actuator 130 are used to move and track the OCT probe 120 accurately. The robotics automates the OCT scan path planning and execution, therefore, achieves fast, comprehensive kidney monitoring without requiring human input. A particular configuration employs a robotic OCT system with real-time 2D OCT image sampling and OCT image-based probe pose update pipeline. Because the 2D OCT images defining each segment 150′ are streamed while the robot 102 is dynamically optimizing the probe pose at high frequency, every OCT image is comprehensive and of a width to coalesce or “stitch” to the adjacent segment. In addition, depth-encoded mapping (FIG. 7C) and axial attenuation mapping (FIG. 7B) are introduced as visualization techniques for a better understanding of the anatomy of the organ. This imaging allows a determination of a suitable, healthy organ for transplant in a timely manner between harvest and implantation.

The information from the OCT scan information mapped to corresponding locations on the tissue specimen 140 can serve as clinically valuable information to identify the health status of the kidney thoroughly. This can be done via a GUI by displaying the organ's camera view picture overlaid with the robot scan trajectory and the corresponding OCT image side by side. However, configurations herein substantially improve this by extracting the cross-sectional slices 150 of the kidney from individual OCT images and stitch them in 3D to i) demonstrate the OCT probe tracking accuracy; and ii) provide an intuitive spatial representation of the organ. The stitching process contains three steps: i) extract tissue pixels from the OCT image as point cloud data; ii) calculate the real-world tissue pixel positions under the OCT image frame by incorporating their pixel-wise positions and the OCT probe parameter; iii) transform the tissue positions to robot base frame for 3D visualization. While i) can be achieved using the same pixel intensity thresholding described above, ii) and iii) are done jointly to acquire the tissue point cloud [x^{base} y^{base} [symbol] ]^{T}. In the i^{th} collected OCT image. The tissue point cloud is obtained via:

$$[ \begin{matrix}{x}_{i}^{base} \\ {y}_{i}^{base} \\ {z}_{i}^{base} \\ {1}_{1 \times m}\end{matrix} ] = {T}_{OCT}^{base} \cdot [ \begin{matrix}{0}_{1 \times m} \\ - \frac{{w}_{OCT}}{W} \cdot {c}^{OCT} + \frac{{w}_{OCT}}{2} \\ \frac{{h}_{OCT}}{H} \cdot {r}^{OCT} \\ {1}_{1 \times m}\end{matrix} ]$$

where m is the total number of pixels with intensity above the threshold, c^{OCT} and r^{OCT} are the tissue positions in pixel relative to the OCT image frame, W is the width of the OCT image in pixel, h_{OCT} is the axial FOV of the OCT image in millimeters. Finally, the resultant point cloud is randomly downsampled by 10% and spatially denoised to remove the outliers.

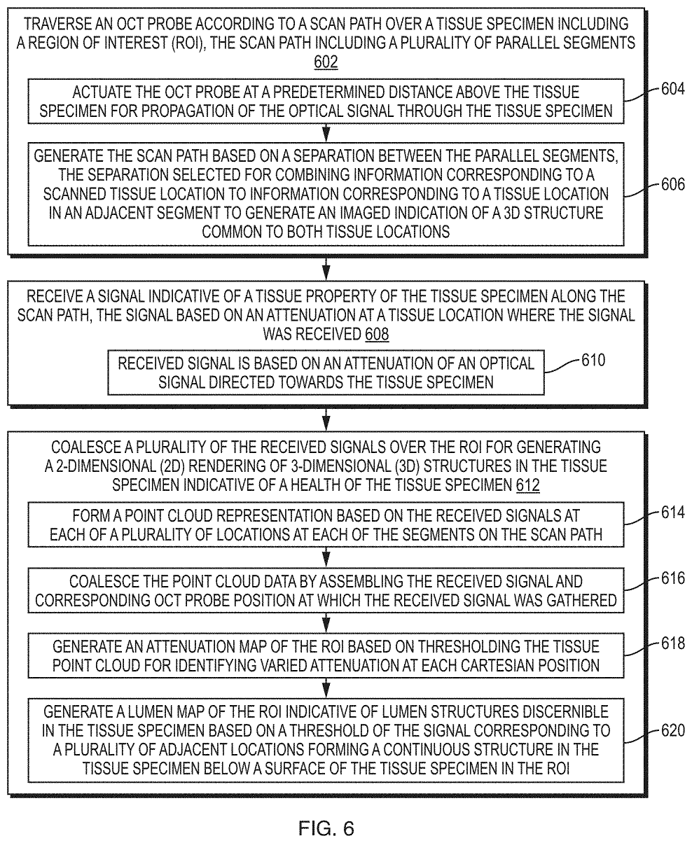

FIG. 6 is a flowchart 600 of system operation for gathering the OCT signals as in FIGS. 1-5 to generate a map of the ROI 132. In the example configuration, depicting pre-transplant health assessment of a human kidney, the method for scanning tissue and rendering a 3D scan includes, at step 602, traversing the OCT probe 120 according to a scan path 134 over a tissue specimen 140 including a region of interest 132, where the scan path 134 includes a plurality of parallel segments 150′. The actuator 130 actuates the OCT probe 120 at the predetermined distance h above the tissue specimen 140 for propagation of the optical signal 121 through the tissue specimen, as depicted at step 604.

This includes generating the scan path 134 based on a separation between the parallel segments 150′, where the separation is selected for combining information corresponding to a scanned tissue location to information corresponding to a tissue location in an adjacent segment to generate an imaged indication of a 3D structure common to both tissue locations, as depicted at step 606. Thus, the adjacent segments 150′ define a width for coalescing or stitching the information from the scan signal to combine with the signal from an adjacent location or segment to compute and render a full image, shown below in FIGS. 7B-7C.

In response to the emitted signal 121, the OCT probe 120 receives a signal indicative of a tissue property of the tissue specimen 140 along the scan path 134, such that the signal is based on an attenuation at a tissue location where the signal was received, as shown at step 608. The received signal is based on an attenuation of the optical signal 121 directed towards the tissue specimen, as the emitted signal is subject to scattering based on the tissue quality and variable penetration of the emitted signal 121, as disclosed at step 610.

From the scan signals, the image processor 124 coalesces a plurality of the received signals over the ROI for generating a 2-dimensional (2D) rendering of 3-dimensional (3D) structures in the tissue specimen indicative of a health of the tissue specimen 140, as depicted at step 612. This includes forming a point cloud 152 representation based on the received signals at each of a plurality of locations at each of the segments 150′ on the scan path 134, as shown at step 614 and FIG. 4A. The received signal includes information of adjacent vertical sections for varying Y-axis 164 positions, and longitudinally along the segment in the X-axis 166 direction as the OCT probe 120 follows the scan path 134. The image processer 124 coalesces the point cloud 152 data by assembling the received signal and corresponding OCT probe position at which the received signal was gathered, as depicted at step 616. The image processor 124 generates an attenuation map of the ROI based on thresholding the tissue point cloud for identifying varied attenuation at each cartesian position, as shown at step 618. Alternatively, or in addition, the image processor 124 generates a lumen map of the ROI indicative of lumen structures discernible in the tissue specimen based on a threshold of the signal corresponding to a plurality of adjacent locations forming a continuous structure in the tissue specimen below a surface of the tissue specimen in the ROI, as depicted at step 620.

FIG. 7A shows the signal received from a scan of a vertical section (“slice”) of FIGS. 3, 4A and 4B. Referring to FIGS. 3-6, scan data or information from a single vertical section 150 results in an intensity map 170, also expressed as an attenuation signal 172. The image processor 124 aggregates the signal 172 received along the segment based on a series of locations along the segment 150′ and corresponding received signals at the respective locations, such that the aggregated signal defines a planar region depicting the vertical section 150 through the tissue specimen parallel to planar regions corresponding to other segments of the plurality of segments 150′, shown in FIG. 3.

FIGS. 7B and 7C show an axial attenuation mapping and a depth encoded mapping, respectively, from the scanning of FIGS. 1-7A. Stitching refers to coalescing each of the vertical sections 150 in a side-by-side arrangements of parallel planes, and rendering a color or grey scale map of the attenuation looking downward at each location on the ROI 134. After the scan, the 2D OCT images of each segment 150′ are analyzed for the presence of anatomical features, which can then be stitched together using pose stamps into 3D visualization then projected into 2D with a continuous colormap. As an example, two types of feature extraction for kidney evaluation are shown, namely an axial attenuation mapping in FIG. 7B, formed by the extinction coefficients extracted from the segments 150′ and depth-encoded mapping in FIG. 7C, formed by the tissue surface depth. The depth-encoded mapping (FIG. 7C) is suitable for illustrating the geometry of the tissue surface. Its colormap smoothness reflects the relative tracking accuracy of the OCT probe and the efficacy of dynamic probe pose optimization. On the other hand, the axial attenuation mapping (FIG. 7B) reveals the microstructures under the tissue surface in individual 2D images.

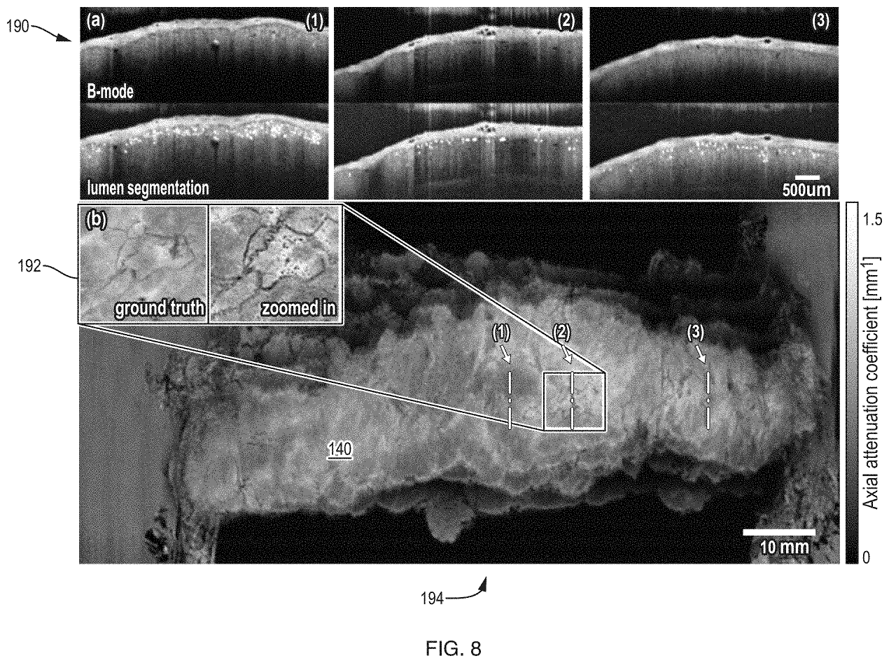

FIG. 8 shows a further example of an axial attenuation mapping of an entire human kidney. Referring to FIGS. 3-8, the capability to cover a large area was demonstrated by imaging an ex-vivo kidney sample for pre-transplant kidney monitoring. The sample specimen 140 was fixed and submerged in formalin to preserve the microstructures, and had a footprint of 106.39 mm-by-37.70 mm. The system imaged the entire kidney, which had a footprint of 123.61 mm-by-53.41 mm, using a path plan consists of 10 scan lines, with 3 mm overlap between consecutive scan lines for the segments 150′. The system collected 40,525 2D OCT images and their corresponding location, which were later used to extract clinically useful information. Such large area comprehensive scan is not possible with conventional commercial OCT imaging systems.

The system can automatically maintain a constant height h of the sample surface 132 in 2D OCT images by regulating the distance between the probe 120 and the specimen 140, hence guaranteeing the quality of individual image. The axial attenuation map is used to verify the localization accuracy of the 2D OCT images. The system generated a whole kidney axial attenuation map 194 by compounding 10 attenuation maps from all the scan lines using the OCT probe's localization information. The central region of the map clearly showed vessel-like microstructures. A smaller area on the kidney (5 mm-by-5 mm) was imaged using a traditional 3D OCT probe and the corresponding ground truth map 192 was generated for comparison. Qualitative comparison between the ground truth and part of the whole kidney attenuation map showed no significant distortion in the shape of the vessel structures, indicating that the OCT probe localization is reliable.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for scanning tissue, comprising:
traversing an OCT probe according to a scan path over a tissue specimen including a region of interest (ROI), the scan path including a plurality of parallel segments;
receiving a signal indicative of a tissue property of the tissue specimen along the scan path, the signal based on an attenuation at a tissue location where the signal was received; and
coalescing a plurality of the received signals over the ROI for generating a 2-dimensional (2D) rendering of 3-dimensional (3D) structures in the tissue specimen indicative of a health of the tissue specimen.

2. The method of claim 1 wherein the received signal is based on an attenuation of an optical signal directed towards the tissue specimen.

3. The method of claim 2 further comprising actuating the OCT probe at a predetermined distance above the tissue specimen for propagation of the optical signal through the tissue specimen.

4. The method of claim 1 further comprising generating the scan path based on a separation between the parallel segments, the separation selected for combining information corresponding to a scanned tissue location to information corresponding to a tissue location in an adjacent segment to generate an imaged indication of a 3D structure common to both tissue locations.

5. The method of claim 1 further comprising aggregating the signal received along the segment based on a series of locations along the segment and corresponding received signals at the respective locations, the aggregated signal defining a planar region through the tissue specimen parallel to planar regions corresponding to other segments of the plurality of segments.

6. The method of claim 1 further comprising
forming a point cloud representation based on the received signals at each of a plurality of locations at each of the segments on the scan path, and
coalescing the point cloud data by assembling the received signal and corresponding OCT probe position at which the received signal was gathered.

7. The method of claim 1 further comprising traversing the tissue specimen by, for each segment on the scan path, commencing a traversal of the segment by actuating the OCT probe for approaching a surface of the tissue specimen on each with a landing movement that disposes the OCT probe downward to a height based on a proportion of a distance above the tissue surface and a height above the bottom tissue surface.

8. The method of claim 5 further comprising:
defining a cartesian volume of the ROI based on the region of interest and an orientation of an actuator directing the traversal of the OCT probe;
forming a tissue point cloud based on the signal received at each location and the position of the OCT probe, a distance of the OCT probe above a surface of the tissue specimen, and a position along the scan path; and
rendering a map indicative of the ROI based on a pixel defined by a cartesian position of the ROI within the cartesian volume.

9. The method of claim 8 further comprising generating an attenuation map of the ROI based on thresholding the tissue point cloud for identifying varied attenuation at each cartesian position.

10. The method of claim 8 further comprising generating a lumen map of the ROI indicative of lumen structures discernible in the tissue specimen based on a threshold of the signal corresponding to a plurality of adjacent locations forming a continuous structure in the tissue specimen below a surface of the tissue specimen in the ROI.

11. A medical scanning device, comprising:
an OCT probe for traversing a scan path over a tissue specimen including a region of interest (ROI), the scan path including a plurality of parallel segments;
a robotic arm for actuating the OCT probe at a predetermined distance above the tissue specimen for propagation of the optical signal through the tissue specimen;
an image processor connected to the OCT probe for receiving a signal indicative of a tissue property of the tissue specimen along the scan path, the signal based on an attenuation at a tissue location where the signal was received; and
coalescing a plurality of the received signals over the ROI for generating a 2-dimensional (2D) rendering of 3-dimensional (3D) structures in the tissue specimen indicative of a health of the tissue specimen.

12. The method of claim 11 wherein the received signal is based on an attenuation of an optical signal directed towards the tissue specimen.

13. The method of claim 11 wherein the scan path is based on a separation between the parallel segments, the separation selected for combining information corresponding to a scanned tissue location to information corresponding to a tissue location in an adjacent segment to generate an imaged indication of a 3D structure common to both tissue locations.

14. The method of claim 11 wherein the image processor is configured to aggregate the signal received along the segment based on a series of locations along the segment and corresponding received signals at the respective locations, the aggregated signal defining a planar region through the tissue specimen parallel to planar regions corresponding to other segments of the plurality of segments.

15. The method of claim 11 wherein the image processor includes a memory configured for forming a point cloud representation based on the received signals at each of a plurality of locations at each of the segments on the scan path, the image processor configured for coalescing the point cloud data by assembling the received signal and corresponding OCT probe position at which the received signal was gathered.

16. The method of claim 11 wherein the robotic arm drives an actuator for traversing the tissue specimen by, for each segment on the scan path, commencing a traversal of the segment by actuating the OCT probe for approaching a surface of the tissue specimen on each with a landing movement that disposes the OCT probe downward to a height based on a proportion of a distance above the tissue surface and a height above the bottom tissue surface.

17. The method of claim 14 wherein the image processor is further configured to:
define a cartesian volume of the ROI based on the region of interest and an orientation of an actuator directing the traversal of the OCT probe;
form a tissue point cloud based on the signal received at each location and the position of the OCT probe, a distance of the OCT probe above a surface of the tissue specimen, and a position along the scan path; and
render a map indicative of the ROI based on a pixel defined by a cartesian position of the ROI within the cartesian volume.

18. The method of claim 17 further comprising a visual display having a rendered attenuation map of the ROI based on thresholding the tissue point cloud for identifying varied attenuation at each cartesian position.

19. The method of claim 17 further comprising a visual display having a rendered lumen map of the ROI indicative of lumen structures discernible in the tissue specimen based on a threshold of the signal corresponding to a plurality of adjacent locations forming a continuous structure in the tissue specimen below a surface of the tissue specimen in the ROI.

20. A computer program embodying program code on a non-transitory computer readable storage medium that, when executed by a processor, performs steps for implementing a method for scanning tissue, the method comprising:
traversing an OCT probe according to a scan path over a tissue specimen including a region of interest (ROI), the scan path including a plurality of parallel segments;
receiving a signal indicative of a tissue property of the tissue specimen along the scan path, the signal based on an attenuation at a tissue location where the signal was received; and
coalescing a plurality of the received signals over the ROI for generating a 2-dimensional (2D) rendering of 3-dimensional (3D) structures in the tissue specimen indicative of a health of the tissue specimen.
